(19) United States
(12) Patent Application Publication
(10) Pub. No.: US 2016/0304479 A1
(43) Pub. Date: Oct. 20, 2016

(54) SULFONATES OF FURAN-2,5-DIMETHANOL AND (TETRAHYDROFURAN-2,5-DIYL)DIMETHANOL AND DERIVATIVES THEREOF

(71) Applicant: ARCHER DANIELS MIDLAND COMPANY, Decatur, IL (US)

(72) Inventor: Kenneth Stensrud, Decatur, IL (US)

(21) Appl. No.: 15/038,061

(22) PCT Filed: Dec. 12, 2014
(86) PCT No.: PCT/US14/70012

§ 371 Date: May 20, 2016

Related U.S. Application Data
(60) Provisional application No. 61/918,217, filed on Dec. 19, 2013.

(51) Int. Cl. C07D 307/42; C07D 307/52; C07F 9/54; C07D 307/24; C07D 307/10; C07D 307/44; C07D 307/12

(52) U.S. Cl. CPC C07D 307/42; C07D 307/44; C07D 307/52; C07D 307/12; C07D 307/24; C07D 307/10; C07F 9/5456

(57) ABSTRACT

A process for preparing furanic mono- and/or di-sulfonate molecules from the reduction products of HMF, in particular, from either 2,5-bis-hydroxymethyltetrahydrofurans (THF-diols) or furan-2,5-dimethanol (FDM) under relatively mild conditions is described. The process involves reacting THF-diols or FDM with at least a sulfonate species, and a reagent of either 1) a nucleophilic base or 2) combination of a non-nucleophilic base and a nucleophile, as two separate reagents. The furanic sulfonates synthesized according to the process and some of the associated compounds that can be derivatized from the sulfonates are also provided.

### BENEFIT OF PRIORITY

The present application claims benefit of priority of U.S. Provisional Application No. 61/918,217, filed on Dec. 19, 2013, the contents of which are incorporated herein by reference.

### FIELD OF INVENTION

The present disclosure relates to certain cyclic bi-functional materials that are useful as monomers in polymer synthesis, as well as intermediate chemical compounds. In particular, the present invention pertains to furanic sulfonate molecules, particular methods by which such molecules are prepared, and certain derivative compounds or materials that incorporate these molecules.

### BACKGROUND

Biomass contains carbohydrates or sugars (i.e., hexoses and pentoses) that can be converted into value added products. Bio-based fuels are an example of an application with growing interest. Another application of interest is the use of biomass as feedstock for synthesis of various industrial chemicals from renewable hydrocarbon sources. In recent years, an increasing effort has been devoted to find ways to utilize biomass as feedstock for the production of organic chemicals because of its abundance, renewability, and worldwide distribution.

Organic compounds that are readily derived from sugars include furans, robust cyclic ethers that possess structural features that can be useful for making certain polymers, pharmaceuticals, or solvents, among other industrial constituents. A related compound that has received considerable attention of late is 5-(hydroxymethyl)furfural (HMF), a major dehydration product of fructose, an abundant, inexpensive monosaccharide (Scheme 1).

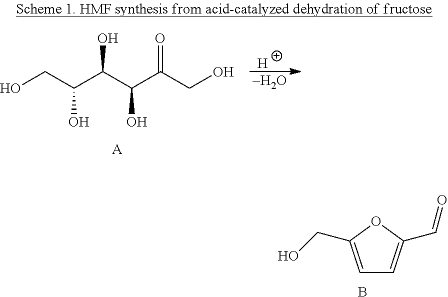

HMF is a versatile chemical antecedent to various furanic ring-based derivatives that are known intermediates for a multitude of chemical syntheses, and are plausible surrogates for aromatic hydrocarbons that derive from petroleum resources. Due to the diverse functionalities of HMF, some have proposed that HMF be used to produce a wide range of commodities such as polymers, solvents, surfactants, pharmaceuticals, and plant protection agents. As substitutes, derivatives of HMF are comparable to benzene-based aromatic compounds or to other compounds containing a furan or tetrahydrofuran (THF). HMF and 2,5-disubstituted furans and THF analogs, therefore, have great potential in the field of intermediate chemicals from renewable agricultural resources.

In order to compete with petroleum-based derivatives, however, preparation of HMF derivatives from common agricultural source materials, such as sugars, must be economical. Until recently, furanics have not been commercialized because large-scale production of furanic intermediates has not been cost-effective. The common dehydration route of fructose to HMF generates many side products, making subsequent purification severely cumbersome yet indispensable. Various different processes have been advanced for the catalytic conversion of sugar to furan chemicals. (See generally, X. Tong et al., “Biomass into Chemicals: Conversion of Sugars to Furan Derivatives by Catalytic Processes,” APPLIED CATALYSIS A: GENERAL 385 (2010) 1-13.)

HMF itself, however, is rather unstable and tends to polymerize or decompose under thermo-oxidative conditions with prolonged storage at ambient conditions. Thus, one should look to derivatives of HMF for practical commercial utility. Two derivatives of interest are: furan-2,5-dimethanol (abbreviated as FDM) and 2,5-bis-(hydroxymethyl)-tetrahydrofuran (abbreviated as bHMTHF), also known colloquially as THF-diols, presented in Scheme 2.

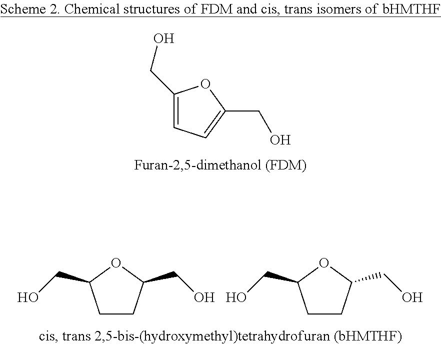

FDM is produced from partial hydrogenation (aldehyde reduction) of HMF (Scheme 3), while bHMTHF
is a saturated analog produced in a 9:1 cis to trans diastereometic ratio when both the ring and aldehyde moieties of HMF are reduce completely (Scheme 4). (See e.g., U.S. Pat. Nos. 7,317,116, or 7,393,963 B2.) These materials can be of value as molecular antecedents, for example, to polyesters, polyurethane foams, plasticizers, resins, surfactants, dispersants, lubricants, agricultural chemicals, or as a solvents, binders, or humectants.

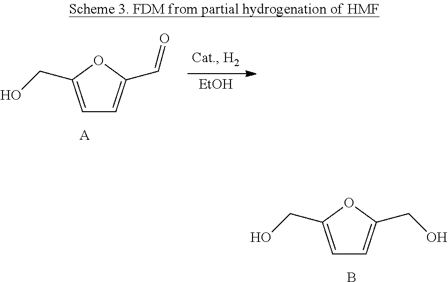

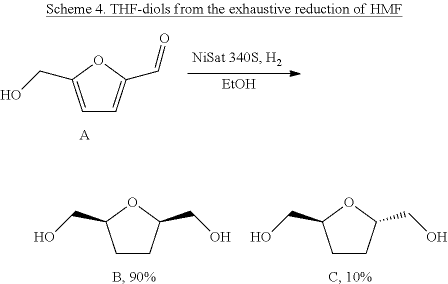

To become market competitive with petroleum products, however, the preparation of HMF derivatives from standard agricultural raw materials, such as sugars, need to become economically feasible in terms of cost. Heretofore, research for chemical derivatives using FDM and/or bHMTHFs has received limited attention due in part to the great cost and relative paucity (e.g., ˜$200 per gram commercially) of the compounds. Recently, a need has arisen for a way to unlock the potential of FDM and bHMTHFs and their derivative compounds, as these chemical entities have gained attention as valuable glycolic antecedents for the preparation of polymers, solvents, additives, lubricants, and plasticizers, etc. Furthermore, the inherent, immutable chirality of bHMTHFs makes these compounds useful as potential species for pharmaceutical applications or candidates in the emerging chiral auxiliary field of asymmetric organic synthesis. Given the potential uses, a cost efficient and simple process that can synthesis derivatives from FDM and/or bHMTHFs would be appreciated by manufacturers of both industrial and specialty chemicals alike as a way to better utilize biomass-derived carbon resources.

### SUMMARY OF THE INVENTION

The present invention relates in part to a method for making furanic sulfonate molecules from the reduction products of HMF, in particular, the preparation of sulfonates from either a) furan-2,5-dimethanol (FDM) or b) 2,5-bis-(hydroxymethyl)-tetrahydrofuran (bHMTHF). The method involves: contacting a reduction product of 5-(hydroxymethyl)furfural (HMF) with at least a sulfonate species and a reagent of either 1) a nucleophilic base or 2) a combination of a non-nucleophilic base and a nucleophile.

In certain embodiments of the present method, one may contact either (THF)-diol or (FDM) with at least a sulfonate species, and a reagent of either 1) a nucleophilic base, or 2) a combination of a non-nucleophilic base and a nucleophile, to prepare, respectively, at least a 1) THF bismethylene a) mono- and/or b) disulfonates compound; or at least a 2) a furan bismethylene a) mono and/or b) disulfonate compound.

In another aspect, the present invention pertains to the mono- and disulfonate compounds made from the synthesis process described herein. Embodiments include, for example, THF-bismethylene monosulfonate, THF-bismethylene disulfonate, furan-bismethylene monosulfonate, and furan-bismethylene disulfonate.

In yet another aspect, the present invention discloses various primary or secondary derivative compounds that can be synthesized from either 1) THF-diol or 2) FDM, or their corresponding THF or FDM 1a, 2a) monosulfonates and/or 1b, 2b) disulfonates as a starting or precursor material for various chemical reactions. Such derivative materials can be useful as either substitutes for existing compounds or new chemical building blocks in various uses.

Additional features and advantages of the present synthesis process and material compounds will be disclosed in the following detailed description. It is understood that both the foregoing summary and the following detailed description and examples are merely representative of the invention, and are intended to provide an overview for understanding the invention as claimed.

### DETAILED DESCRIPTION OF THE INVENTION

### Section I.—Description

Derived from HMF, 2,5-bis-(hydroxymethyl)-tetrahydrofurans (bHMTHF, also known as THF-diols) and furan-2,5-dimethanol (FDM) hold considerable potential as a precursor monomers for polymers, softeners, adhesives, humectants, resins, dispersants, plasticizers, building blocks for surfactants, and agricultural chemicals. The corresponding bismethylene mono- and disulfonates of tetrahydrofuran and furan, respectively, permit facile preparation of template-orientated targets to be achieved via supervening, straightforward nucleophilic displacement transformations.

The present disclosure provides, in part, an efficient and facile process for synthesizing tetrahydrofuran-2,5-bismethylene (THF) sulfonates and furan-2,5-bismethylenes (FDM) sulfonates under relatively mild conditions. The process involves reacting THF-diols or FDM with at least a sulfonate species, and a reagent of either 1) a nucleophilic base or 2) combination of a non-nucleophilic base and a nucleophile (e.g., triethylamine (TEA)), as two separate reagents. One can use a variety of sulfonates, such as mesylate (methanesulfonate),

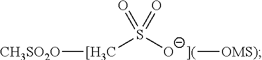

triflate (trifluoromethanesulfonate),

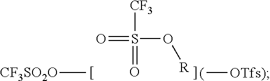

tosylate (p-toluenesulfonate),

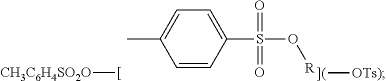

esylate (ethanesulfonate),

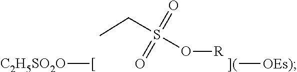

besylate (benzenesulfonate), C₆H₅SO₂O—

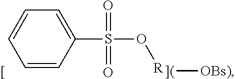

or other sulfonate species without limitation. Examples of nucleophilic bases can include, without limitation: pyrimidine, dimethyl-aminopyridine, imidazole, pyrrolidine, and morpholine. Examples of non-nucleophilic bases can include, but are not restricted to, hindered amines, triethylamine, diisopropylethylamine, dibutylamine, carbonate salts, such as sodium and potassium carbonate, bicarbonate salts, such as sodium and potassium bicarbonate, and acetate salts, such as sodium or potassium acetate.

According to an embodiment, the process involves reacting THF-diols or FDM with an alkyl or aryl sulfonyl chloride or anhydride employing a nucleophilic base in an organic solvent at room temperature or below. This is illustrated in Scheme 5, a) for THF and b) for FDM respectively.

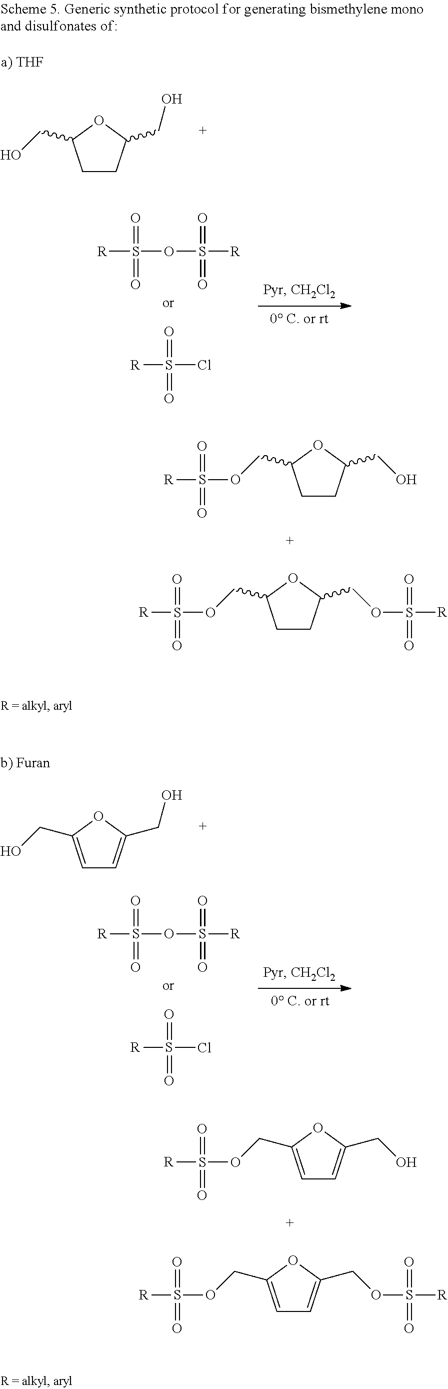

The present synthesis process can result in satisfactory yields of corresponding THF and FDM bismethylene mono and/or disulfonates, as demonstrated in the accompanying examples. The process is able to produce THF and FDM bismethylene mono and disulfonates in reasonably high molar yields of at least 50% from the THF-diol and FDM starting materials, typically about 55% or 60%-70% or 75%). With proper control of the reaction conditions and time, one can achieve a yield of about 80%-90% or better of these materials.

Schemes 6-8 present some examples of THF-sulfonate species that may be produced according to the present process. Scheme 6 shows the isomers of THF-monotriflates; Scheme 7, shows the isomers of THF-monomesylates; and Scheme 8 shows THF ditriflates.

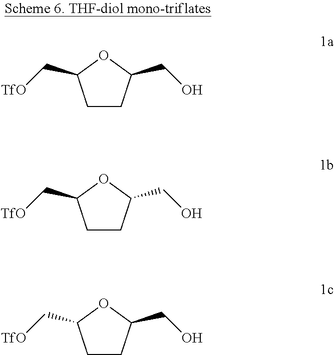 [SMILES: O=S(=O)(OC[C@@H]1CC[C@H](CO)O1)C(F)(F)F ; O=S(=O)(OC[C@@H]1CC[C@@H](CO)O1)C(F)(F)F ; O=S(=O)(OC[C@H]1CC[C@H](CO)O1)C(F)(F)F]

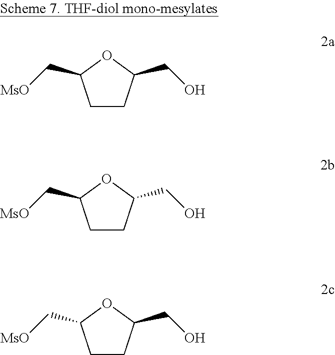 [SMILES: CS(=O)(=O)OC[C@@H]1CC[C@H](CO)O1 ; CS(=O)(=O)OC[C@@H]1CC[C@@H](CO)O1 ; CS(=O)(=O)OC[C@H]1CC[C@H](CO)O1]

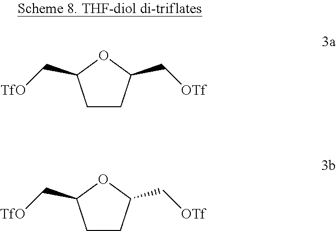 [SMILES: O=S(=O)(OC[C@H]1CC[C@@H](COS(=O)(=O)C(F)(F)F)O1)C(F)(F)F ; O=S(=O)(OC[C@@H]1CC[C@@H](COS(=O)(=O)C(F)(F)F)O1)C(F)(F)F]

In certain embodiments, the sulfonate is preferably a triflate as it manifests the highest nucleofugacity (>10⁶) of any of the other sulfonates, thus permitting the supervening displacements to be conducted at reduced temperatures (room temperature or lower) and concomitantly lowering the likelihood for side product formation. The overall reaction exhibits relatively fast kinetics and is posited to operate through a transitory, activated triflate complex intermediate. The reaction is usually conducted at a low temperature, 0-25° C. (e.g., typically about −10° C. or −12° C. to about −20° C. or −25° C.), to control the reaction kinetics more easily and lessen the chances for side product formation. This reaction is essentially irreversible, as the liberated triflate is entirely non-nucleophilic, subsequently serving as a mere spectator salt. The role of the nucleophilic base is to form a complex with the triflate, this posited to augment the reactivity with the FDM or THF-diols. The subsequent products formed are a THF or furan bismethylene mono- or di-triflate depending on the number of sulfonyl equivalents added, simultaneously releasing nucleophilic base, which then deprotonates the alkoxonium intermediate.

Though not as powerful as triflate, tosylate, mesylate, brosylate, benzenesulfonate, ethylsulfonate or other sulfonate species are copacetic nucleofuges, particularly when deployed at higher temperatures, with the capacity to achieve overall yields that commensurate triflates. These sulfonates tend to react more slowly, however, in comparison to the triflate. To compensate for this, operations at higher temperatures are typically needed for better yields when using these other species.

For purpose of illustration, the following discussion will involve triflates as the sulfonate moiety, but the general principles herein will apply equally to the other sulfonate species. Bismethylene triflates of FDM are illustrated in Scheme 9.

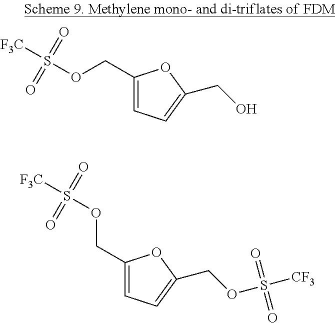 [SMILES: O=S(=O)(OCc1ccc(CO)o1)C(F)(F)F ; O=S(=O)(OCc1ccc(COS(=O)(=O)C(F)(F)F)o1)C(F)(F)F]

These materials are potentially versatile precursors to an array of subsequent compounds, such as thioethers, amines, halides, alkyl/aryl chain extensions, all achieved by nucleophilic displacement reactions, as adumbrated in Scheme 5 with FDM bismethylene ditriflate.

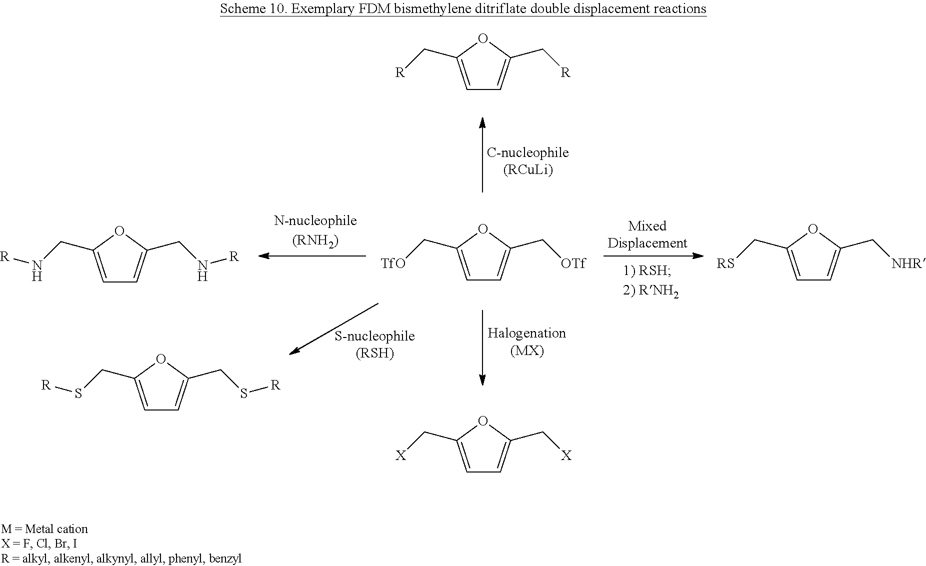

The synthesis of derivative compounds from THF mono- and di-triflates are analogous and deducible, mutandis mutatis, from the preceding FDM models, as the degree of reactivity for analogous cis and trans bismethylene mono and ditriflates of THF, shown in Scheme 11 (monotriflates) and Scheme 12 (ditriflates), respectively, can be the same as for the aforementioned FDM bismethylene.

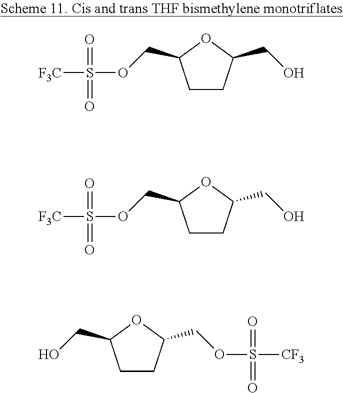 [SMILES: O=S(=O)(OC[C@@H]1CC[C@H](CO)O1)C(F)(F)F ; O=S(=O)(OC[C@@H]1CC[C@@H](CO)O1)C(F)(F)F ; O=S(=O)(OC[C@@H]1CC[C@@H](CO)O1)C(F)(F)F]

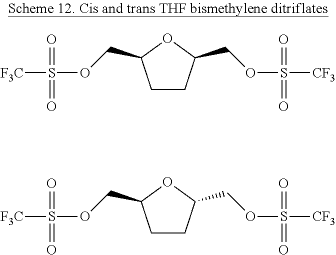 [SMILES: O=S(=O)(OC[C@H]1CC[C@@H](COS(=O)(=O)C(F)(F)F)O1)C(F)(F)F ; O=S(=O)(OC[C@@H]1CC[C@@H](COS(=O)(=O)C(F)(F)F)O1)C(F)(F)F]

The reactivity of the mono- and ditriflates has the potential to open synthesis to an assortment of useful thio-ethers, amines, halides through straightforward single and double displacement reactions. Examples of such synthesis with THF mono- and di-triflates as the sulfonate are presented in Scheme 13, and Scheme 14, respectively.

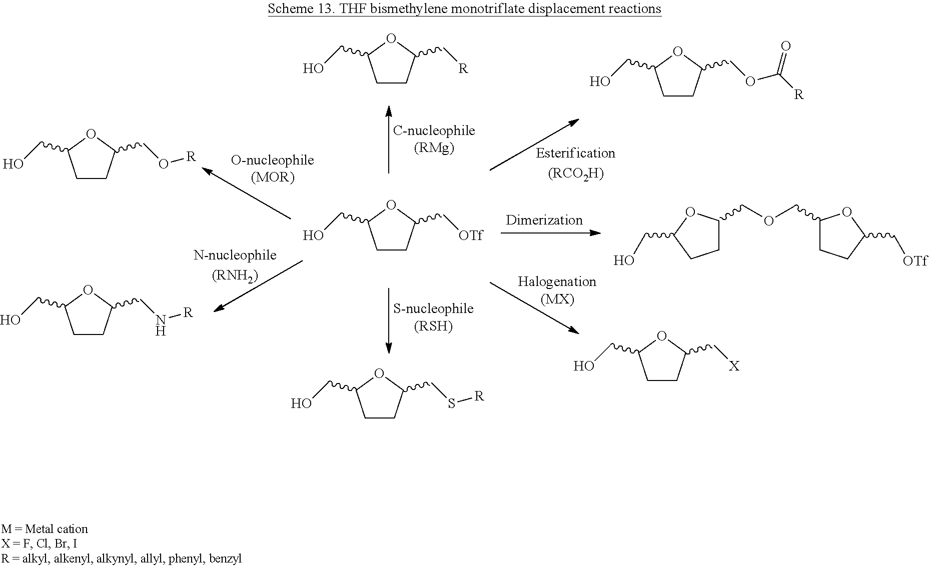

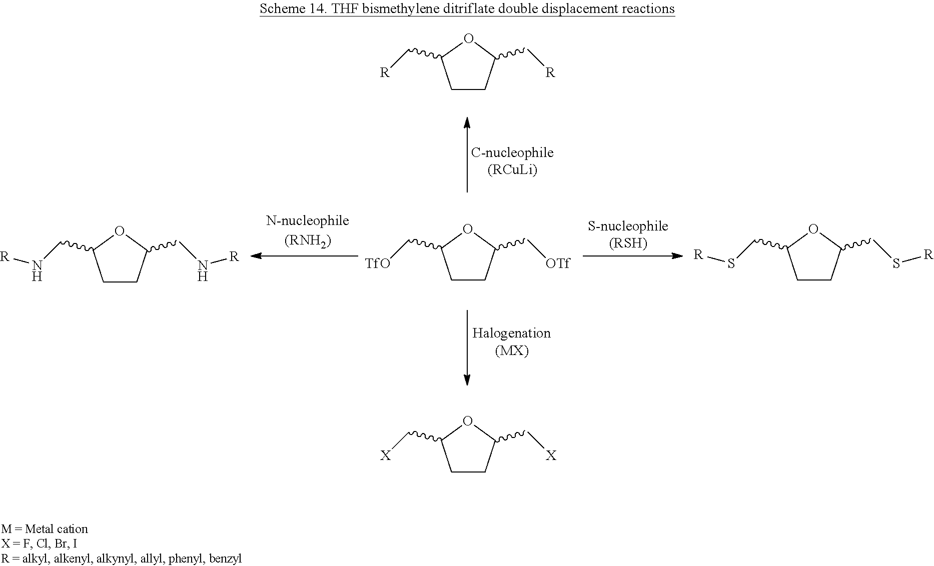

A further illustration of the intrinsic sulfonate versatility is in Scheme 15, which underscores derivitizations of the alcohol moiety with concomitant preservation of the sulfonate.

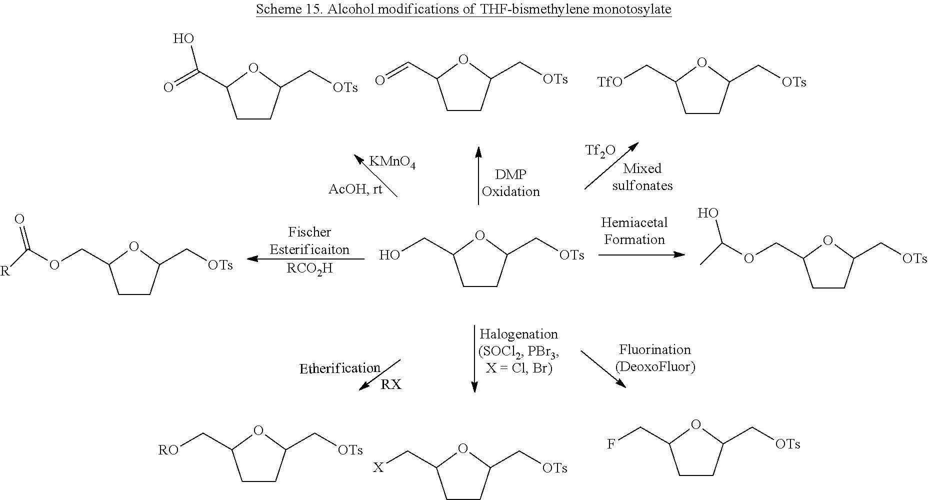

Particular illustrative examples of derivative compounds that can be made from both FDM and THF-sulfonates are presented in the associated examples that follow.

### Section II.—Examples

The following examples are provided as illustration of the different aspects of the present disclosure, with the recognition that altering parameters and conditions, for example by change of temperature, time and reagent amounts, and particular starting species and catalysts and amounts thereof, can affect and extend the full practice of the invention beyond the limits of the examples presented.

The following examples refer to mesylates, triflates, and tosylates for purposes of illustration; however, the scope of the invention is not necessarily limited to those particular embodiments, since others may incorporate other more common or commercially available sulfonate species.

### A. Tetrahydrofuran Bismethylene Mono and Ditriflates

### Example 1

### Synthesis of ((2S,5R)-5-(hydroxymethyl)tetrahydrofuran-2-yl)methyl trifluoromethanesulfonate 1a, ((2S,5S)-5-(hydroxymethyl)tetrahydrofuran-2-yl)methyl trifluoromethanesulfonate 1b, ((2R,5R)-5-(hydroxymethyl)tetrahydrofuran-2-yl)methyl trifluoromethanesulfonate 1c

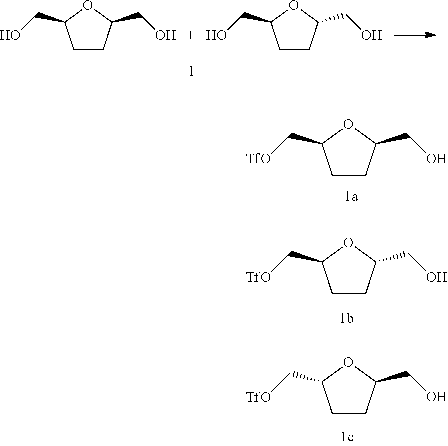 [SMILES: OC[C@@H]1CC[C@H](CO)O1 ; OC[C@@H]1CC[C@@H](CO)O1 ; O=S(=O)(OC[C@@H]1CC[C@H](CO)O1)C(F)(F)F ; O=S(=O)(OC[C@@H]1CC[C@@H](CO)O1)C(F)(F)F ; O=S(=O)(OC[C@H]1CC[C@H](CO)O1)C(F)(F)F]

### Experimental

An oven dried, 25 mL single-neck round bottomed flask equipped with a ½″×⅛″ tapered PTFE coated magnetic stir bar was charged with 212 mg of THF-diols 1 (1.60 mmol), 400 μL of pyridine (˜3 eq.) and 10 mL of anhydrous methylene chloride. The neck was capped with a rubber septum and a needle affixed to an argon inlet and the flask immersed in a saturated brine/ice bath (−10° C.). While stirring and under an argon blanket, 270 μL of triflic anhydride (1.60 mmol) was added dropwise over a 10 minute period. After complete addition, the flask was removed from the ice bath, warmed to ambient temperature, and the reaction continued for 2 more hours. After this time, an aliquot was removed and a portion spotted on a silica gel thin-layer chromatography plate abutting a spot from the THF diol starting material for comparison. The plate was developed using a 100% ethyl acetate eluent, and after staining with cerium molybdate, the product mixture revealed three distinct spots manifesting Rf₁=0.67 (THF bismethylene ditriflate), Rf₂=0.32 (THF bismethylene monotriflates), and R_{f}=0 (unreacted THF-diols). The reaction was concluded at this time and the residue then poured directly onto a prefabricated silica gel column, where gradient flash chromatography with hexanes/ethyl acetate eluent afforded 141 mg of THF bismethylene monotriflates 1a-c (1:1 hexanes/ethyl acetate) as a pale yellow oil (33% of theoretical) after concentration. ¹H NMR (400 MHz, CDCl₃, cis isomer) δ (ppm) 4.26 (m, 1H), 3.96-3.94 (m, 2H), 3.85-3.83 (m, 2H), 3.70 (m, 1H), 3.65 (m, 1H), 1.93 (m, 2H), 1.66 (m, 2H); ¹³C NMR (100 MHz, CDCl₃, cis isomer) δ (ppm) 120.4, 88.9, 84.6, 74.1, 65.1, 30.6, 30.0.

### Example 2

### Synthesis of ((2S,5R)-5-(hydroxymethyl)tetrahydrofuran-2-yl)methyl methanesulfonate 2a, ((2S,5S)-5-(hydroxymethyl)tetrahydrofuran-2-yl)methyl methanesulfonate 2b, ((2R,5R)-5-(hydroxyl-methyl)tetrahydrofuran-2-yl)methyl methanesulfonate 2c

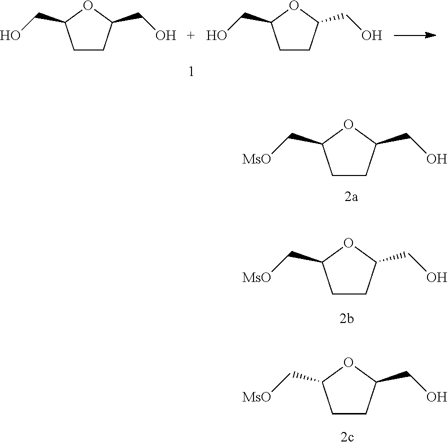 [SMILES: OC[C@@H]1CC[C@H](CO)O1 ; OC[C@@H]1CC[C@@H](CO)O1 ; CS(=O)(=O)OC[C@@H]1CC[C@H](CO)O1 ; CS(=O)(=O)OC[C@@H]1CC[C@@H](CO)O1 ; CS(=O)(=O)OC[C@H]1CC[C@H](CO)O1]

### Experimental

An oven dried, 25 mL single-neck round bottomed flask equipped with a ½″×⅛″ tapered PTFE coated magnetic stir bar was charged with 212 mg of THF-diols 1 (1.60 mmol), 400 μL of pyridine (˜3 eq.) and 10 mL of anhydrous methylene chloride. The neck was capped with a rubber septum and a needle affixed to an argon inlet and the flask immersed in a saturated brine/ice bath (−10° C.). While stirring and under an argon blanket, 125 μL of mesyl chloride (1.60 mmol) was added dropwise over a 10 minute period. After complete addition, the flask was removed from the ice bath, warmed to ambient temperature, and the reaction continued for 2 more hours. After this time, an aliquot was removed and a portion spotted on a silica gel thin-layer chromatography plate abutting a spot from the THF diol starting material for comparison. The plate was developed using a 100% ethyl acetate eluent, and after staining with cerium molybdate, the product mixture revealed two distinct spots manifesting Rf₁=0.58 (THF bismethylene dimesylates), Rf₂=0.24 (THF bismethylene monomeslylates), and Rf₃=0 (unreacted THF-diols). The reaction was halted at this time, and the solution then poured directly onto a prefabricated silica gel column, where gradient flash chromatography with hexanes/ethyl acetate eluent afforded 124 mg of THF bismethylene monomesylates 2a-c (1:1.5 hexanes/ethyl acetate) as a colorless oil (37% of theoretical) after concentration. ¹H NMR (400 MHz, CDCl₃, cis isomer) δ (ppm) 4.22 (m, 1H), 3.92-3.89 (m, 2H), 3.81-3.79 (m, 2H), 3.67 (m, 1H), 3.61 (m, 1H), 3.22 (s, 1H), 1.91 (m, 2H), 1.63 (m, 2H); ¹³C NMR (100 MHz, CDCl₃, cis isomer) δ (ppm) 88.4, 83.1, 73.0, 65.1, 39.2, 30.4, 29.6.

### Example 3

### Synthesis of ((2R,5S)-tetrahydrofuran-2,5-diyl)bis(methylene) bis(trifluoromethanesulfonate) 3a, and ((2S,5S)-tetrahydrofuran-2,5-diyl)bis(methylene) bis(trifluoromethanesulfonate) 3b

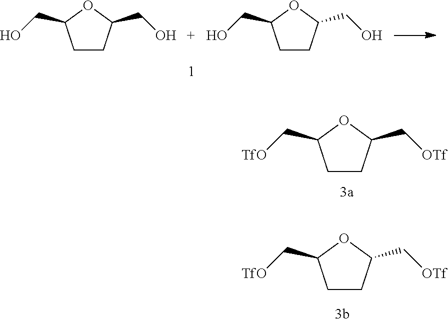 [SMILES: OC[C@@H]1CC[C@H](CO)O1 ; OC[C@@H]1CC[C@@H](CO)O1 ; O=S(=O)(OC[C@H]1CC[C@@H](COS(=O)(=O)C(F)(F)F)O1)C(F)(F)F ; O=S(=O)(OC[C@@H]1CC[C@@H](COS(=O)(=O)C(F)(F)F)O1)C(F)(F)F]

### Experimental

An oven dried, 25 mL single-neck round bottomed flask equipped with a ½″×⅛″ tapered PTFE coated magnetic stir bar was charged with 226 mg of THF-diols 1 (1.71 mmol), 410 μL of pyridine (˜3 eq.) and 10 mL of anhydrous methylene chloride. The neck was capped with a rubber septum and a needle affixed to an argon inlet and the flask immersed in a saturated brine/ice bath (−10° C.). While stirring and under an argon blanket, 574 μL of triflic anhydride (3.42 mmol) was added dropwise over a 15 minute period. After complete addition, the flask was removed from the ice bath, warmed to ambient temperature, and the reaction continued for 2 more hours. After this time, an aliquot was removed and a portion spotted on a silica gel thin-layer chromatography plate abutting a spot from the THF diol starting material for comparison. The plate was developed using a 100% ethyl acetate eluent, and after staining with cerium molybdate, the product mixture revealed one distinct spot, Rf₁=0.67 (THF-diol ditriflate). The solution was then poured directly onto a prefabricated silica gel column, where gradient flash chromatography with hexanes/ethyl acetate eluent and cerium molybdate visualization afforded 457 mg of title compounds 3a and 3b as a light brown oil (67% of theoretical) after concentration. ¹H NMR (400 MHz, CDCl₃, cis isomer) δ (ppm) 4.58 (m 2H), 4.47 (m, 2H), 4.44 (m, 2H), 4.32 (m, 2H), 2.15 (m, 2H), 1.87 (m, 2H); ¹³C NMR (100 MHz, CDCl₃, cis isomer) δ (ppm) 120.2, 84.1, 70.4, 30.7.

### B. Derivatives of Bismethylene Mono and Disulfonates of THF

### I. Bismethylene THF Monosulfonate Analogs

### Example 1

### Preparation of 4-(((2S,5R)-5-(hydroxymethyl)tetrahydrofuran-2-yl)methoxy)-4-oxobutan-1-aminium 2,2,2-trifluoroacetate 3a and stereoisomers, 3b, c

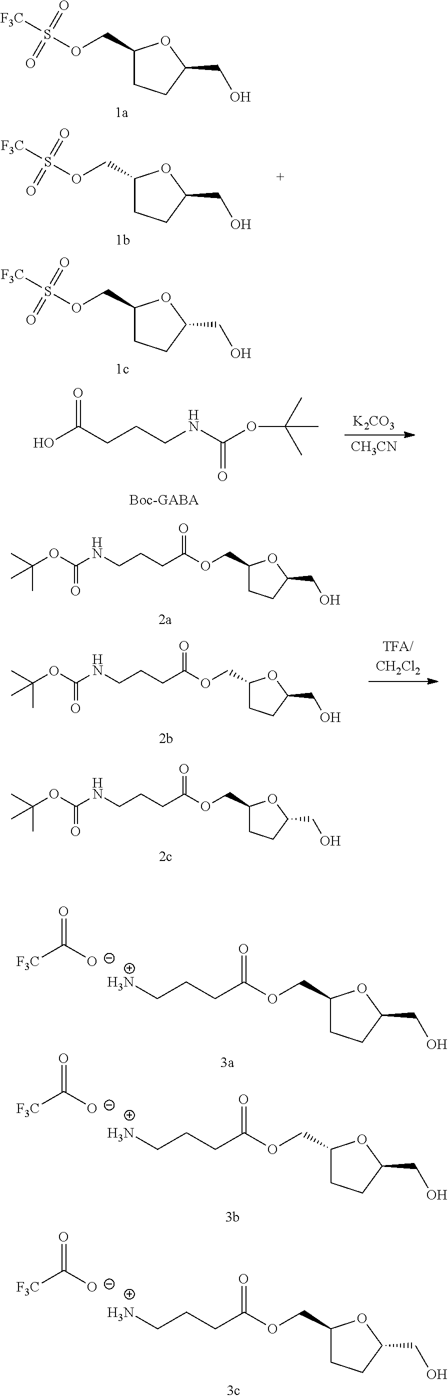

Part 1: Synthesis of ((2S,5R)-5-(hydroxymethyl)tetrahydrofuran-2-yl)methyl 4-((tert-butoxycarbonyl)-amino)-butanoate 2a and stereoisomers 2b, c.

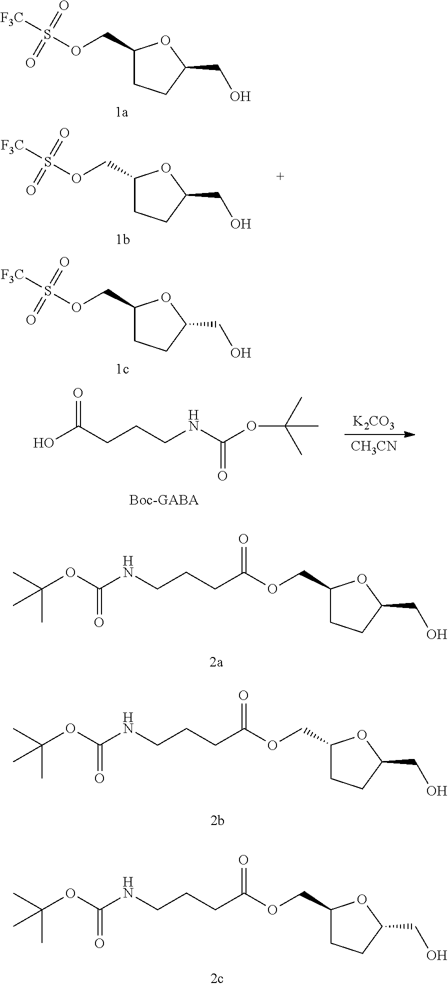

### Experimental

A single neck, 25 ml round bottomed flash equipped with a PTFE coated magnetic stir bar was charged with 225 mg of THF-diol monotriflate 1 (9:1 dr=meso:R,R, S,S; 0.851 mmol), 193 mg of itoc-GABA (1.02 mmol), 353 mg of K₂CO₃ (2.553 mmol) and 15 mL of anhydrous acetonitrile. A reflux condenser was outfitted to the flask and, while vigorously stirring, the solution brought to reflux. Aliquots were removed at 1 h increments and analyzed by TLC (100% ethyl acetate) with visualization using cerium molybdate. After 12 hours, the band associated with 1 (Rf=0.46) was shown to have disappeared in favor of two overlapping bands with Rf=0.33, 0.31 respectively), specifying the reaction had culminated. The residual solids were then filtered with a medium porosity sintered glass funnel and filtrate concentrated in vacuo, affording 243 mg of a transparent semi-solid. From TLC, this material was assumed to be mostly 2a-d and used in the supervening step without further analysis or purification.

Part 2: Synthesis of 3-(((2S,5S)-5-(hydroxymethyl)tetrahydrofuran-2-yl)methoxy)-3-oxopropan-1-amin-ium 2,2,2-trifluoroacetate 3a and stereoisomers, 3b, c.

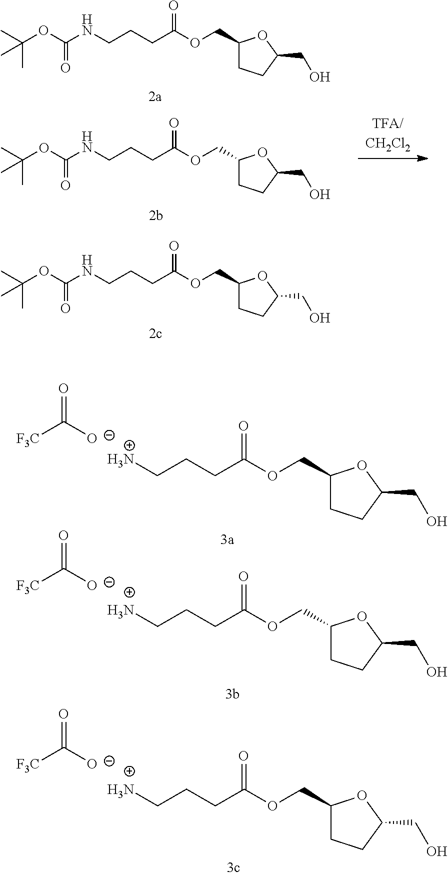

### Experimental

A single neck, 10 mL round bottomed flask equipped with a PTFE coated magnetic stir bar was charged with 243 mg of 2a-c (0.801 mmol) and 5 mL of 50% trifluoroacetic acid in CH₂Cl₂. While vigorously stirring, effervescence was immediately observed (CO₂ loss), which continued for 5 minutes then attenuated. After this time excess solvent was removed, the resultant colorless oil dissolved in 5 mL of water, frozen and lyophilized overnight, furnishing 235 mg of 3a-c as a light yellow solid (92% of theoretical). ¹H NMR (400 MHz, D₂O, cis isomer) δ (ppm) 4.56 (m, 2H), 4.51 (m, 1H), 4.23 (m, 1H), 3.86 (m, 2H), 3.74 (m, 1H), 3.57 (t, J=1.2 Hz, 2H), 3.01 (t, J=6.6 Hz, 2H), 2.03 (m, 2H), 1.51 (m, 2H); ¹³C NMR (100 MHz, D₂O, cis isomer) δ (ppm) 171.8, 162.6, 112.2, 87.1, 86.5, 81.7, 80.3, 67.8, 62.7, 60.3, 38.2, 33.6, 31.6, 30.8, 29.0, 27.6.

### Example 2

### Preparation of sodium (2S,5R)-5-(((methylsulfonyl)oxy)methyl)tetrahydrofuran-2-carboxy-late and stereoisomers 4a-c

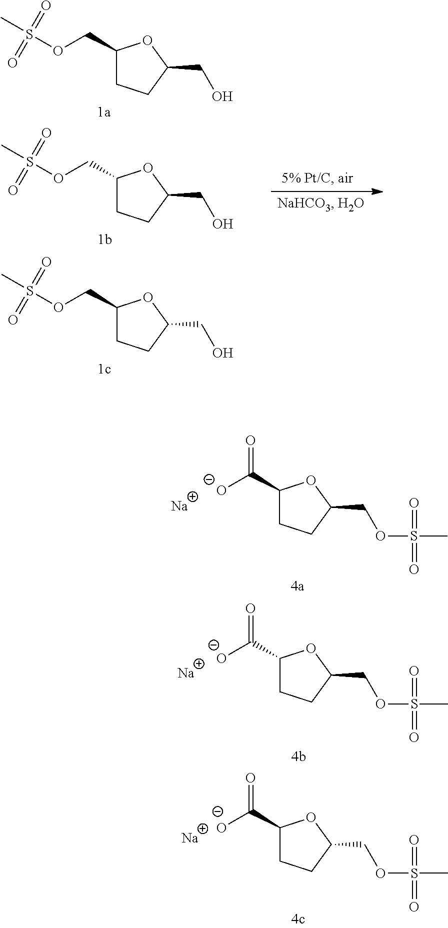

### Experimental

A single neck, 100 mL round bottomed flask equipped with a magnetic stir bar was charged with 500 mg of THF-diol mono-mesylate (2.32 mmol) 1, 474 mg of 5% Pt/C (200 g/mol of 1), 1.20 g of NaHCO₃ (14.27 mmol) and 60 mL of deionized water. The neck of the flask was then capped with a rubber septum and an air inlet affixed via an 18 gauge stainless needle whose beveled tip was positioned near the bottom of the heterogeneous solution. In addition, six 2 inch, 16 gauge needles pierced the septum, utilized as air vents. While stirring, the flask was immersed in an oil bath and heated at 60° C. with vigorous sparging of air for a 24 hour time period. After this time, the Pt/C was removed by filtration and the aqueous residue analyzed by silica gel thin layer chromatography using 100% ethyl acetate developing solution and cerium molybdate stain for spot illumination. A single band, positioned at the baseline, was observed while that for 1 (0.54 with an authentic sample) was absent, suggesting that 1 had been fully converted to the mono-sodium salt. A single band was observed at the baseline. Cogent proof for the conversion of 1 arose from a clean ¹³C NMR (100 MHz, D₂O, cis isomer) spectrum that manifested salient signals at 177.4, 88.9, 83.4, 67.1, 30.2, 26.6 ppm.

### Example 3 and 4

### Direct Mono-Halogenations of Bismethylene THF Sulfonates—Viable Organomagnesium (Grignard), Organocopper, Organozinc (Reformatsky), and Organolithium Precursors

### Example 3

### Synthesis of ((2S,5S)-5-(chloromethyl)tetrahydrofuran-2-yl)methyl 4-methylbenzene-sulfonate and isomers, B

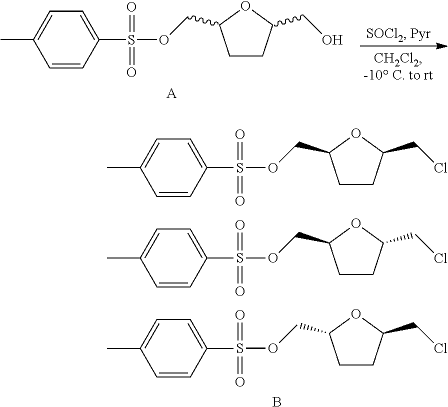

### Experimental

A single necked, 25 mL round bottomed flask equipped with a PTFE coated Teflon magnetic stir bar was charged with 200 mg of 5-(hydroxymethyl)tetrahydrofuran-2-yl)methyl 4-methylbenzenesulfonate A (0.698 mmol), 1 mL of pyridine (12.4 mmol), and 10 mL of anhydrous methylene chloride. The neck was stoppered with a rubber septum and an argon inlet connected via a 16″ needle and the flask immersed in a saturated brine/ice bath that measured approximately −10° C. While stirring and under argon, 56 μL of thionyl chloride (0.768 mmol) was added dropwise over 15 min via syringe. After addition, the mixture was stirred for another 2 hours at this temperature, after which the ice was removed, and the mixture stirred overnight. Excess solvent, pyridine and unreacted thionyl chloride were then removed under reduced pressure, affording a brown oil that was dissolved in a minimum amount of methylene chloride and poured onto a prefabricated silica gel column. Gradient flash chromatography using hexanes/ethyl acetate as eluents afforded 102 mg of ((2S,5R)-5-(chloromethyl)tetrahydrofuran-2-yl)methyl 4-methylbenzenesulfonate and isomers B (48% of theoretical) as a pale yellow oil after concentration. ¹H NMR (400 MHz, CDCl₃, cis isomer) δ (ppm) 7.81 (d, J=8.2 Hz, 2H), 7.39 (d, J=8.2 Hz, 2H), 4.26 (m, 1H), 4.11 (m, 1H), 3.95-3.53 (m, 2H), 3.71 (m, 1H), 3.40 (m, 1H), 2.41 (s, 3H), 1.98 (m, 2H), 1.75 (m, 2H); ¹³C NMR (100 MHz, CDCl₃, cis isomer) δ (ppm) 146.2, 140.9, 131.5, 129.4, 83.0, 81.1, 72.8, 31.3, 30.5, 22.8.

### Example 4

### Synthesis of ((2S,5S)-5-(bromomethyl)tetrahydrofuran-2-yl)methyl 4-methylbenzene-sulfonate and isomers, B

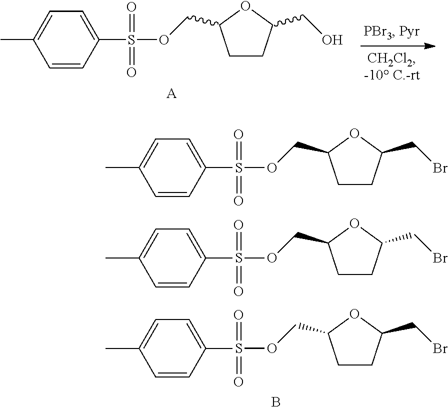

### Experimental

A single necked, 25 mL round bottomed flask equipped with a PTFE coated Teflon magnetic stir bar was charged with 200 mg of 5-(hydroxymethyl)tetrahydrofuran-2-yl)methyl 4-methylbenzenesulfonate A (0.698 mmol), 1 mL of pyridine (12.4 mmol), and 10 mL of anhydrous methylene chloride. The neck was stoppered with a rubber septum and an argon inlet connected via a 16″ needle and the flask immersed in a saturated brine/ice bath that measured approximately −10° C. While stirring and under argon, 72 μL of phosphorus tribromide (0.768 mmol) was added dropwise over 30 min using a syringe. After addition, the mixture was stirred for another 2 hours at this temperature, after which the ice was removed, and the mixture stirred overnight. Excess phosphorus tribromide was quenched with a few drops of water, and residual solvent and pyridine were then removed under reduced pressure, affording a reddish oil that was dissolved in a minimum amount of methylene chloride and poured onto a prefabricated silica gel column. Gradient flash chromatography using hexanes/ethyl acetate as eluents afforded 81 mg of ((2S,5S)-5-(bromomethyl)tetrahydrofuran-2-yl)methyl 4-methylbenzenesulfonate and isomers B (33% of theoretical) as a colorless oil after concentration. ¹H NMR (400 MHz, CDCl₃, cis isomer) δ (ppm) 7.81 (d, J=8.2 Hz, 2H), 7.41 (d, J=8.2 Hz, 2H), 4.27 (m, 1H), 4.14 (m, 1H), 3.95-3.53 (m, 2H), 3.59 (m, 1H), 3.28 (m, 1H), 2.40 (s, 3H), 1.98 (m, 2H), 1.74 (m, 2H); ¹³C NMR (100 MHz, CDCl₃, cis isomer) δ (ppm) 146.2, 141.0, 131.5, 129.5, 82.8, 80.3, 40.2, 31.3, 30.5, 22.8.

### Example 5

### Synthesis of ((2R,5S)-5-(((ethylsulfonyl)oxy)methyl)tetrahydrofuran-2-yl)methylhexanoate and isomers B

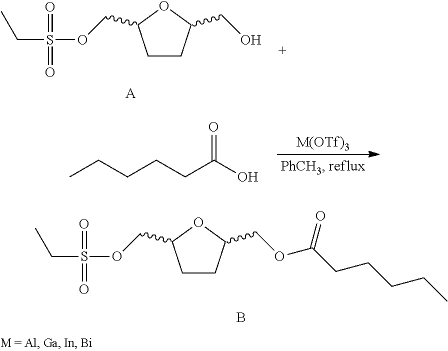

### Experimental

A single necked, 50 mL round bottomed flask equipped with a Teflon coated magnetic stir bar was charged with 250 mg of A (1.11 mmol), hexanoic acid (1.22 mmol), 40 mg of indium triflate (0.055 mmol), and 25 mL of toluene. The flask was then outfitted with a Dean-Stark apparatus and while vigorously stirring, the mixture was brought to reflux, spanning for 24 h. After this time, the solids were filtered, and organic residue washed with saturated sodium bicarbonate, then removed. The withdrawn aqueous phases were combined and extracted with one 10 mL volume of toluene. The toluene layers were integrated, dried with anhydrous magnesium sulfate and evaporated under reduced pressure, affording a yellow gum. This material was then dissolved in a minimal amount of methylene chloride and charged to a pre-packed silica gel column, where flash chromatography with a 0 to 25% ethyl acetate in hexanes eluent and cerium molybdate visualization furnished 211 mg (59% of theoretical) of B and stereoisomers (TLC R_{f}˜0.42, 40% ethyl acetate in hexanes) after concentration. ¹H NMR (400 MHz, CDCl₃, cis isomer) δ (ppm) 4.44-4.42 (m, 2H), 4.18 (m, 1H), 4.05 (m, 1H), 3.99 (m, 1H), 3.77 (m, 1H), 3.36 (q, 2H), 2.45 (t, J=6.2 Hz, 2H), 1.91 (m, 2H), 1.67 (m, 2H), 1.61 (m, 2H), 1.33-1.29 (m, 7H), 0.87 (t, 0.7=5.2 Hz, 3H); ¹³C NMR (100 MHz, CDCl₃, cis isomer) δ (ppm) 172.1, 84.2, 83.6, 82.8, 81.4, 73.6, 62.8, 47.1, 35.0, 33.8, 33.2, 31.3, 30.2, 23.7, 14.6, 8.3. The order of catalyst activity for screened metal triflates, according to yields of B, were as follows: Al<In<Bi<Sn<Ga with corresponding yields of 49%, 59%, 72%83%, 91% respectively.

### Example 6

### Synthesis of ((2S,5R)-5-formyltetrahydrofuran-2-yl)methylbenzenesulfonate and isomers, B

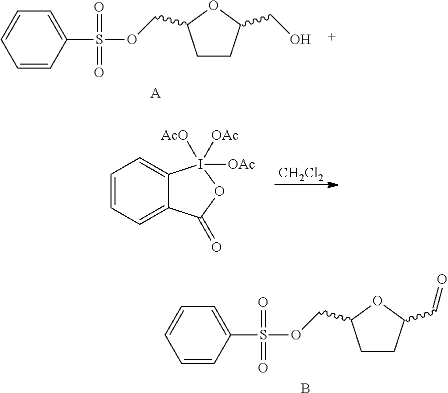

### Experimental

A single necked, 25 mL round bottomed flask equipped with a teflon coated magnetic stir bar was charged with 250 mg of A (0.918 mmol), 400 mg of DMP (0.922 mmol), and 10 mL of methylene chloride. The mixture was stirred vigorously for 4 h, after which time an aliquot was withdrawn and analyzed by H NMR (400 MHz, CDCl₃) and ¹³C NMR (100 MHz, CDCl₃). Both spectra evinced intense, high frequency signals at 9.54 and 200.1 ppm respectively, adducing the presence of an aldehyde. Solids were filtered and the permeate charged to a prefabricated silica gel column, where gradient flash chromatography with hexanes/ethyl acetate and UV-Vis illumination furnished 111 mg of B as a white solid (45% of theoretical) after concentration. ¹H NMR analysis (400 MHz, CDCl₃, cis isomer) δ (ppm) 9.54 (s, 1H), 8.11 (m, 1H), 7.78-7.75 (m 4H), 4.55 (m, 1H), 4.19 (m, 1H), 3.94-3.92 (m, 2H), 2.19 (m, 1H), 2.00-1.98 (m, 2H), 1.73 (m, 1H); ¹³C NMR (100 MHz, CDCl₃, cis isomer) δ (ppm) 200.1, 150.3, 135.2, 131.1, 129.3, 96.0, 83.8, 73.3, 28.5, 26.9.

### II. Single and Double Displacement Variants of THF Bismethylene Disulfonates

### Example 1

### Synthesis of (2R,2′R)-3,3′-((((2R,5S)-tetrahydrofuran-2,5-diyl)bis(methylene))bis(sulfane-diyl))bis(2-acet-amidopropanoic acid) and isomers, B

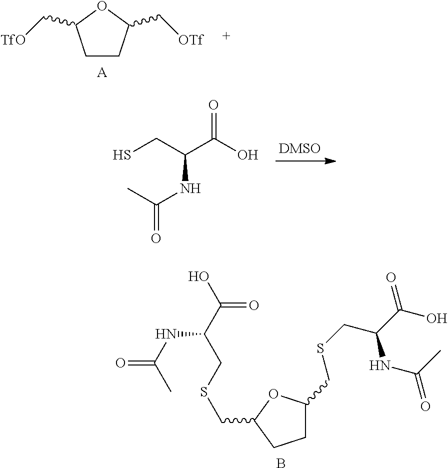

### Experimental

A dry, 10 mL round bottomed flask equipped with a magnetic stir bar was charged with 100 mg of A (0.252 mmol), 83 mg of N-acetyl-L-cysteine (0.504 mmol), 500 μL of triethylamine and 7 mL of dry DMSO. The mixture was stirred at room temperature for 72 h. After this time, the excess solvent removed by vacuum distillation and the resultant beige solid dissolved in a minimum amount of acetone, then charged to a pre-fabricated silica gel column, where gradient flash chromatography with hexanes/ethyl acetate/acetone as eluents and cerium molybdate visualization afforded the title compound B as a beige solid weighing 41 mg (39% of theoretical) after concentration. ¹H NMR (400 MHz, d⁶-DMSO, cis isomer) δ (ppm) 4.88 (t, J=6.6 Hz, 2H), 4.00 (m, 2H), 3.01 (m, 2H), 2.77 (m, 2H), 2.60 (m, 2H), 2.35 (m, 2H), 1.99 (d, J=8.2 Hz, 2H), 1.90 (s, 6H), 1.61 (d, J=8.1 Hz, 2H); ¹³C NMR (100 MHz, d⁶-DMSO, cis isomer) δ (ppm) 146.4, 141.3, 129.1, 128.0, 127.2, 108.2, 57.2, 51.3, 43.1, 33.0, 32.7, 21.7.

### Example 2

### Synthesis of (2R,5S)-2,5-bis(fluoromethyl)tetrahydrofuran and isomers, B

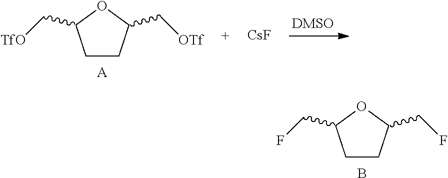

### Experimental

A dry, 10 mL round bottomed flask equipped with a magnetic stir bar was charged with 100 mg of A (0.252 mmol), 112 mg of CsF (0.756 mmol) and 5 mL of dry DMSO. The mixture was stirred at room temperature for 24 h. After this time, the solution was transferred to a 50 mL separatory flask, diluted with 10 mL of methylene chloride and 10 mL of water, which partitioned satisfactorily. The organic layer was removed, and aqueous layer extracted with three 5 mL volumes of methylene chloride. The combined organic phases were concentrated under reduced pressure producing a brown oil. This material was dissolved in a minimum amount of methylene chloride, and charged to a prefabricated silica gel column, where gradient flash chromatography with hexanes/ethyl acetate eluent and cerium molybdate visualization furnished the title compound B as a pale yellow oil, weighing 30 mg (88% of theoretical) after concentration. ¹H NMR (400 MHz, CDCl₃, cis isomer) δ (ppm) 4.34 (m, 2H), 4.06-4.02 (m, 4H), 1.99 (m, 2H), 1.59 (m, 2H); ¹³C NMR (100 MHz, CDCl₃, cis isomer) δ (ppm) 89.1, 80.3, 30.4.

### Example 3 Exhibits a Practicable Synthetic Route Towards Wittig (Phosphonium) Salts of the Title Compounds

### Example 3

### Synthesis of (((2R,5S)-5-(((methylsulfonyl)oxy)methyl)tetrahydrofuran-2-yl)methyl)triphen-ylphosphonium methanesulfonate and isomers B

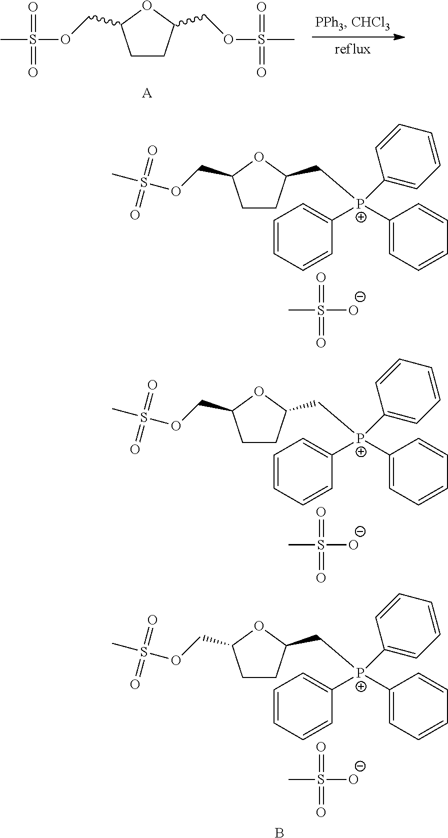

### Experimental

A single necked, 10 mL round bottomed flask equipped with a PTFE coated magnetic stir bar was charged with 50 mg of (tetrahydrofuran-2,5-diyl)bis(methylene) dimethanesulfonate (0.173) mmol), 45 mg of triphenylphosphine (0.173 mmol), and 5 mL of anhydrous chloroform. The flask was outfitted with a condenser, and while stirring, the solution heated to reflux overnight. After this time, the resultant intensely yellow solution was cooled to room temperature and diluted with 5 mL of anhydrous diethyl ether. The addition of ether induced precipitation of a white solid, which was filtered, and washed with 10 mL more ether. After drying overnight, the colorless plates ascribed to B, weighed 86 mg (90% of theoretical). An analytical sample was obtained by recrystallization with ethanol/diethyl ether (1:3). ¹H NMR (400 MHz, d⁶-DMSO, cis isomer) δ (ppm) 7.46-7.44 (m, 15H), 4.20 (m, 1H), 3.91-3.89 (m, 2H), 3.85 (m, 1H), 3.77 (m, 1H), 2.50 (m, 2H), 1.92 (m, 2H), 1.62 (m, 2H); ¹³C NMR (100 MHz, d⁶-DMSO, cis isomer) δ (ppm) 136.1, 133.7, 132.9, 119.6, 83.1, 80.2, 70.9, 54.2, 46.1, 38.9, 32.1, 30.7.

### C. FDM Bismethylene Mono and Disulfonates

### Example 1

### Synthesis of (5-(hydroxymethyl)furan-2-yl)methyl trifluoromethanesulfonate B

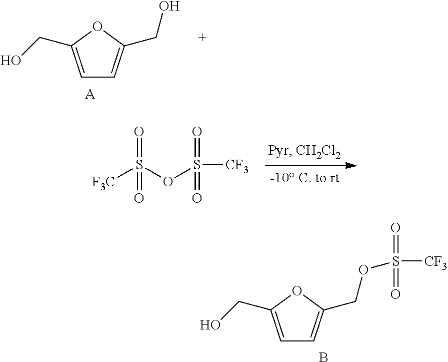

### Experimental

An oven dried, 25 mL single-neck round bottomed flask equipped with a ½″×⅛″ tapered PTFE coated magnetic stir bar was charged with 250 mg of FDM A (1.95 mmol), 472 μL of pyridine (˜3 eq.) and 10 mL of anhydrous methylene chloride. The neck was capped with a rubber septum and a needle affixed to an argon inlet and the flask immersed in a saturated brine/ice bath (−10° C.). While stirring and under an argon blanket, 328 μL of triflic anhydride (1.95 mmol) was added dropwise over a 10 minute period via syringe. After complete addition, the flask was removed from the ice bath, warmed to ambient temperature, and the reaction continued for 3 more hours. After this time, an aliquot was removed and a portion spotted on a silica gel thin-layer chromatography plate abutting a spot from the THF diol starting material for comparison. The plate was developed using a 100% ethyl acetate eluent, and after staining with cerium molybdate, the product mixture revealed three distinct spots manifesting Rf₁=0.63 (FDM di-triflate), Rf₂=0.30 (FDM mono-triflates), and R_{f}=0 (unreacted FDM). The reaction was concluded at this time and residual solution poured directly onto a pre-fabricated silica gel column, where gradient flash chromatography with hexanes/ethyl acetate as the eluent and cerium molybdate visualization produced 182 mg of (5-(hydroxymethyl)furan-2-yl)methyl trifluoromethane-sulfonate B as a light beige solid (36% of theoretical). ¹H NMR (400 MHz, CDCl₃) δ (ppm) 6.38 (d, J=8.4 Hz, 1H), 6.32 (d, J=8.4 Hz), 4.77 (s, 2H), 4.48 (s, 2H), 3.70 (broad, 1H); ¹³C NMR (100 MHz, CDCl₃) δ (ppm) 155.0, 152.8, 119.2, 109.4, 108.6, 70.4, 65.2.

### Example 2

### Synthesis of (5-hydroxymethyl)furan-2-yl)methyl-4-methylbenzene-sulfonate, B

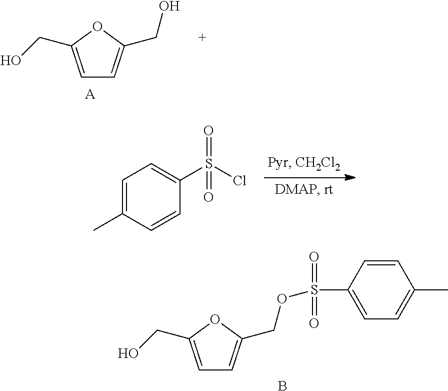

### Experimental

An oven dried, 25 mL single-neck round bottomed flask equipped with a ½″×⅛″ tapered PTFE coated magnetic stir bar was charged with 300 mg of FDM A (2.34 mmol), 566 μL of pyridine (˜3 eq.), 3 mg of DMAP (1 mol %), 446 mg of p-toluenesulfonyl chloride (2.34 mmol) and 10 mL of anhydrous methylene chloride. The homogeneous mixture was stirred for 4 more hours. After this time, an aliquot was removed and a portion spotted on a silica gel thin-layer chromatography plate abutting a spot from the THF-diol starting material for comparison. The plate was developed using a 100% ethyl acetate eluent and product mixture revealed three distinct spots by UV-Vis manifesting Rf₁=0.69 (FDM di-tosylate), Rf₂=0.31 (FDM mono-tosylate), and R_{f}=0 (unreacted FDM). The reaction was concluded at this time and residual solution poured directly onto a pre-fabricated silica gel column, where gradient flash chromatography with hexanes/ethyl acetate as the eluent and UV-Vis illumination produced 279 mg of (5-(hydroxymethyl)furan-2-yl)methyl 4-methylbenzenesulfonate B as a light beige solid (42% of theoretical). H NMR (400 MHz, CDCl₃) δ (ppm) 7.51 (d, J=9.0 Hz, 2H), 7.40 (d, J=9.0 Hz), 6.36 (d, J=8.4 Hz, 1H), 6.31 (d, J=8.4 Hz), 4.68 (s, 2H), 4.35 (s, 2H), 3.70 (broad, 1H), 2.42 (s, 3H); ¹³C NMR (100 MHz, CDCl₃) δ (ppm) 155.0, 152.8, 141.5, 140.2, 132.0, 127.6, 119.2, 109.4, 108.6, 64.1, 8.9, 22.5.

### Example 3

### Synthesis of furan-2,5-diylbis(methylene) bis(trifluoromethanesulfonate) B

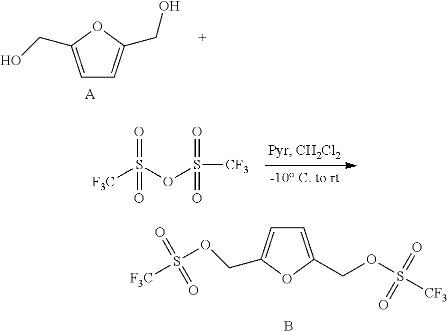

### Experimental

An oven dried, 25 mL single-neck round bottomed flask equipped with a ½″×⅛″ tapered PTFE coated magnetic stir bar was charged with 200 mg of FDM A (1.56 mmol), 378 μL of pyridine (˜3 eq.) and 10 mL of anhydrous methylene chloride. The neck was capped with a rubber septum and a needle affixed to an argon inlet and the flask immersed in a saturated brine/ice bath (−10° C.). While stirring and under an argon blanket, 550 μL of triflic anhydride (3.28 mmol) was added dropwise over a 15 minute period via syringe. After complete addition, the flask was removed from the ice bath, warmed to ambient temperature, and the reaction continued for 2.5 more hours. After this time, an aliquot was removed and a portion spotted on a silica gel thin-layer chromatography plate abutting a spot from the FDM starting material for comparison. The plate was developed using a 100% ethyl acetate eluent, and after staining with cerium molybdate, the product mixture revealed one distinct spots, Rf₁=0.63 (FDM ditriflate). No band was observed at the baseline (R_{f}=0), indicating that all the FDM had been converted. H NMR (CDCl₃, 400 MHz) δ (ppm) 6.42 (s, 2H), 4.81 (s, 4H); ¹³C NMR (CDCl₃, 400 MHz) δ (ppm) 154.71, 120.22, 108.91, 64.02.

### Example 4

### Synthesis of furan-2,5-diylbis(methylene) dimethanesulfonate B

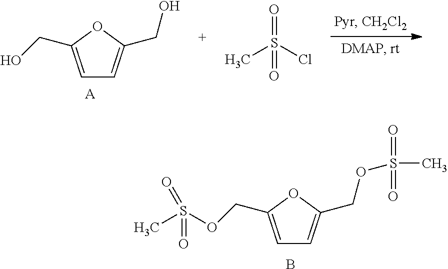

### Experimental

An oven dried, 25 mL single-neck round bottomed flask equipped with a ½″×⅛″ tapered PTFE coated magnetic stir bar was charged with 225 mg of FDM A (1.76 mmol), 425 μL of pyridine (˜3 eq.), 5 mg of DMAP (2 mol %) and 10 mL of anhydrous methylene chloride. The neck was capped with a rubber septum and a needle affixed to an argon inlet. While stirring and under an argon blanket, 286 μL of mesyl chloride (3.70 mmol) was added dropwise over a 15 minute period via syringe and the reaction continued for 3 more hours. After this time, an aliquot was removed and a portion spotted on a silica gel thin-layer chromatography plate abutting a spot from the FDM starting material for comparison. The plate was developed using a 100% ethyl acetate eluent, and after staining with cerium molybdate, the product mixture revealed one distinct spots, Rf₁=0.57 (FDM dimesylate). No band was observed at the baseline (R_{f}=0), indicating that all the FDM had been converted. ¹H NMR (CDCl₃, 400 MHz) δ (ppm) 6.32 (s, 2H), 4.55 (s, 4H), 3.31 (s, 6H); ¹³C NMR (CDCl₃, 400 MHz) δ (ppm) 152.24, 106.62, 63.77, 39.1.

### D. Derivatives of FDM Bismethylene Mono and Disulfonates

### I. Variants of bismethylene FDM monosulfonates

### Example 1

### Synthesis of (5-((benzylthio)methyl)furan-2-yl)methanol, B

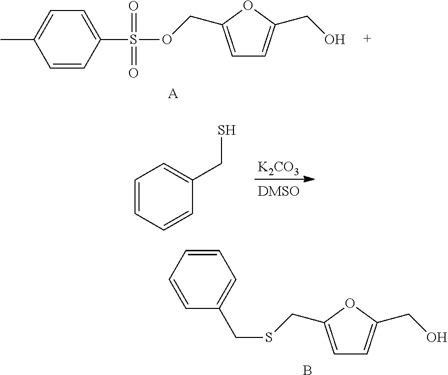

### Experimental

A single necked, 25 mL round bottomed flask equipped with a Teflon magnetic stir bar was charged with 200 mg of (5-(hydroxymethyl)furan-2-yl)methyl 4-methylbenzenesulfonate A (0.708 mmol), 100 μL of benzyl mercaptan (0.850 mmol), 294 mg of potassium carbonate (2.12 mmol) and 10 mL of anhydrous dimethylsulfoxide. The flask was outfitted with a condenser, and while stirring, the mixture heated to 100° C. overnight. After this time, the solution was transferred to a 50 mL separatory funnel and diluted with 10 mL of methylene chloride and 10 mL of water. The organic phase was extracted, washed ×3 with water, then dried with anhydrous sodium sulfate. The residual brown oil was diluted in a minimum amount of methylene chloride and charged to a prefabricated silica gel column, where flash chromatography with hexanes and ethyl acetate as eluents produced 132 mg of (5-((benzylthio)methyl)furan-2-yl)methanol B as a light yellow solid (79% of theoretical). ¹H NMR (CDCl₃, 400 MHz) δ (ppm) 7.48 (d, J=8.0 Hz, 2H), 7.30-7.28 (m, 3H), 6.22 (d, J=7.6 Hz, 1H), 6.08 (d, J=7.6 Hz, 1H), 4.26 (s, 2H), 3.68 (s, 2H), 3.66 (s, 2H), 3.44 (broad, 1H); ¹³C NMR (CDCl₃, 400 MHz) δ (ppm) 152.8, 150.9, 140.5, 129.0, 128.7, 128.0, 109.2, 108.7, 59.0, 34.8, 32.1.

### Example 2

### Preparation of (5-(fluoromethyl)furan-2-yl)methyl methanesulfonate, B

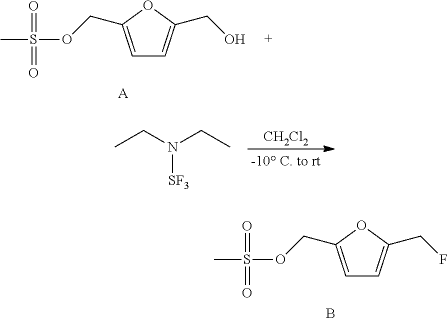

### Experimental

A single necked, 25 mL round bottomed flask equipped with a PTFE coated magnetic stir bar was charged with 300 mg of (5-(hydroxymethyl)furan-2-yl)methyl methanesulfonate A (1.45 mmol) and 10 mL of anhydrous methylene chloride. The flask was then immersed in a saturated brine/ice bath (˜−10° C.) and, while stirring, 384 μL of diethylaminosulfur trifluoride (DAST, 2.91 mmol) was added dropwise over 30 min via syringe. The ice was then removed and mixture continued at room temperature overnight. After that time, a few drops of water were carefully added to quench residual DAST, and the resultant solution poured directly onto a prefabricated silica gel column, where gradient flash chromatography with hexanes/ethyl acetate as the eluents produced 85 mg of (5-(fluoromethyl)furan-2-yl)methyl methanesulfonate B as a colorless oil (28% of theoretical). ¹H NMR (CDCl₃, 400 MHz) δ (ppm) 6.25 (d, J=12 Hz, 2H), 6.00 (d, J=12 Hz, 1H), 5.31 (s, 2H), 4.71 (s, 2H), 3.30 (s, 3H); ¹³C NMR (CDCl₃, 400 MHz) δ (ppm) 152.9, 150.7, 108.6, 107.6, 87.0, 61.2, 40.4.

### II. Variants of Bismethylene FDM Monosulfonates

### Example 1

### Synthesis of N,N′-(furan-2,5-diylbis(methylene))bis(l-phenylmethanamine), B

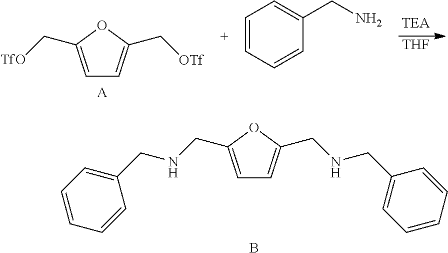

### Experimental

A dry, 10 mL round bottomed flask equipped with a magnetic stir bar was charged with 100 mg of A (0.255 mmol), 56 μL of benzylamine (0.510 mmol), 73 μL of triethylamine (TEA, 0.510 mmol), and 5 mL of dry THF. The flask was attached to a reflux condenser connected to an argon bubbler, and, while vigorously stirring, the mixture was brought 50° C. and maintained overnight. The next morning, the heat was removed, solution cooled to room temperature, and excess solvent removed under high vacuum. The resultant yellow oil was dissolved in a minimum amount of methylene chloride and charged to a prefabricated silica gel column, where gradient flash chromatography with a hexanes/ethyl acetate eluent and UV-Vis illumination afforded the title compound B (eluting with 100% ethyl acetate) as a colorless oil weighing 62 mg (80% of theoretical) after concentration. ¹H NMR (400 MHz, CDCl₃) δ (ppm) 7.36-7.30 (m, 6H), 7.20 (m, 4H), 6.16 (s, 2H), 3.81 (s, 4H), 3.69 (s, 4H); ¹³C NMR (100 MHz, CDCl₃) δ (ppm) 146.4, 141.3, 129.1, 128.0, 127.2, 108.2, 57.2, 51.3.

### Example 2

### Synthesis of 2,5-bis(4-methoxybenzyl)furan, B

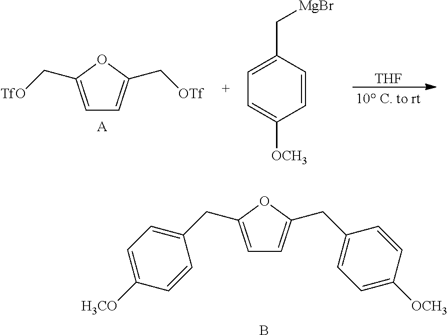

### Experimental

A dry, 10 mL round bottomed flask equipped with a magnetic stir bar was charged with 100 mg of A (0.255 mmol) and 5 mL of dry THF. The flask was immersed in a brine/ice bath (−10° C.), capped with a rubber stopper attached to an argon bubbler, and while stirring and under argon, 510 μL of (4-methoxybenzyl)magnesium bromide (0.510 mmol, 1M in diethyl ether), was added dropwise. After addition, the flask was removed from the ice bath and warmed to room temperature, where stirring persisted for another 1 h. After this time, solids were filtered and excess THF removed under vacuum. The resultant oil was dissolved in a minimum amount of methylene chloride and charged to a prefabricated silica gel column, where gradient flash chromatography employing a hexane/ethyl acetate eluent and UV-Vis illumination afforded 53 mg of the title compound B (eluting at 4:1 hexanes/ethyl acetate) as a light tan solid (68% of theoretical) after concentration. ¹H NMR (400 MHz, CDCl₃) δ (ppm) 7.16 (d, J=9.2 Hz, 2H), 6.82 (d, J=9.2 Hz, 2H), 6.01 (s, 2H), 3.91 (s, 6H), 3.55 (s, 4H); ¹³C NMR (100 MHz, CDCl₃) δ (ppm) 158.2, 155.1, 131.2, 127.7, 112.9, 56.7, 36.9.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

1. A method of preparing a furanic sulfonate compound comprising: contacting a reduction product of 5-(hydroxymethyl)furfural (HMF) with a sulfonate species and a reagent of either 1) a nucleophilic base or 2) a combination of a non-nucleophilic base and a nucleophile.

2. The method according to claim 1, wherein said reduction product of HMF is either a) furan-2,5-dimethanol (FDM) or b) 2,5-bis-(hydroxymethyl)-tetrahydrofuran (bHMTHF).

3. (canceled)

4. (canceled)

5. The method according to claim 1, wherein said sulfonate species is at least one of: mesylate (methanesulfonate), triflate (trifluoromethanesulfonate), tosylate (p-toluenesulfonate), esylate (ethanesulfonate), and besylate (benzenesulfonate).

6. The method according to claim 1, wherein said nucleophilic base is at least: pyrimidine, dimethyl-aminopyridine, imidazole, pyrrolidine, and morpholine.

7. The method according to claim 1, wherein said non-nucleophilic base is at least one of: a hindered amine, triethylamine, diisopropylethylamine, dibutylamine, a carbonate salt, a bicarbonate salt, and an acetate salt.

8. A sulfonate compound prepared according to claim 1, wherein said sulfonate compound is either THF-bismethylene monosulfonate or THF-bismethylene disulfonate.

9. (canceled)

10. A sulfonate compound prepared according to claim 1, wherein said sulfonate compound is either furan-bismethylene monosulfonate or furan-bismethylene disulfonate.

11. (canceled)

12. A primary derivative compound made from THF-bismethylene monosulfonate, the primary derivative compound being selected from at least one of the following groups:
Group I.
a. 2((2R,5S)-5-(hydroxymethyl)-tetrahydrofuran-2-yl)methyl 4-((tert-butoxycarbonyl)amino)-butanoate 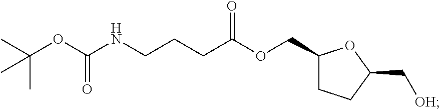 [SMILES: CC(C)(C)OC(=O)NCCCC(=O)OC[C@@H]1CC[C@H](CO)O1]
b. ((2S,5S)-5-(hydroxymethyl)-tetrahydrofuran-2-yl)methyl 4-((tert-butoxycarbonyl)amino)-butanoate 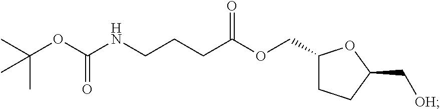 [SMILES: CC(C)(C)OC(=O)NCCCC(=O)OC[C@H]1CC[C@H](CO)O1]
and
c. ((2R,5R)-5-(hydroxymethyl)-tetrahydrofuran-2-yl)methyl 4-((tert-butoxycarbonyl)amino)-butanoate 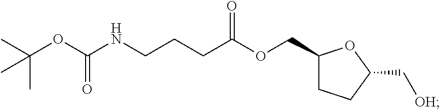 [SMILES: CC(C)(C)OC(=O)NCCCC(=O)OC[C@@H]1CC[C@@H](CO)O1]
Group II.
a. Sodium (2R,5S)-5-((methylsulfonyl)oxy)methyl)tetrahydrofuran-2-carboxylate 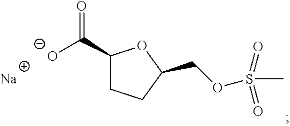
b. Sodium (2S,5S)-5-((methylsulfonyl)oxy)methyl)tetrahydrofuran-2-carboxylate 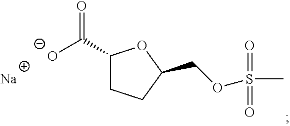
and
c. Sodium (2R,5R)-5-((methylsulfonyl)oxy)methyl)tetrahydrofuran-2-carboxylate 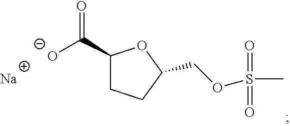
Group III.
a. ((2R,5S)-5-(((ethylsulfonyl)oxy)methyl)tetrahydrofuran-2-yl)methyl hexanoate 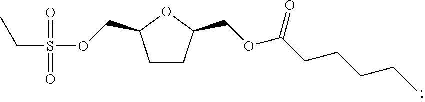 [SMILES: CCCCCC(=O)OC[C@H]1CC[C@@H](COS(=O)(=O)CC)O1]
b. ((2S,5S)-5-(((ethylsulfonyl)oxy)methyl)tetrahydrofuran-2-yl)methyl hexanoate 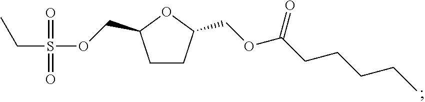 [SMILES: CCCCCC(=O)OC[C@@H]1CC[C@@H](COS(=O)(=O)CC)O1]
and
c. ((2R,5R)-5-(((ethylsulfonyl)oxy)methyl)tetrahydrofuran-2-yl)methyl hexanoate 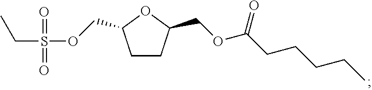 [SMILES: CCCCCC(=O)OC[C@H]1CC[C@H](COS(=O)(=O)CC)O1]
Group IV.
a. ((2S,5R)-5-formyltetrahydrofuran-2-yl)methyl benzenesulfonate 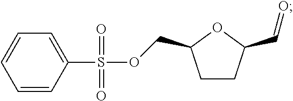 [SMILES: O=C[C@H]1CC[C@@H](COS(=O)(=O)c2ccccc2)O1]
b. ((2S,5S)-5-formyltetrahydrofuran-2-yl)methyl benzenesulfonate 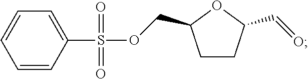 [SMILES: O=C[C@@H]1CC[C@@H](COS(=O)(=O)c2ccccc2)O1]
and
c. ((2R,5R)-5-formyltetrahydrofuran-2-yl)methyl benzenesulfonate 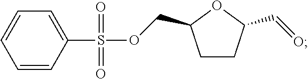 [SMILES: O=C[C@@H]1CC[C@@H](COS(=O)(=O)c2ccccc2)O1]
Group V.
a. (R)-2-amino-3-(((2S,5R)-5-(hydroxymethyl)tetrahydrofuran-2-yl)methyl)thio)propanoic acid 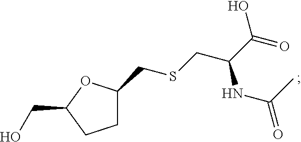 [SMILES: CC(=O)N[C@@H](CSC[C@H]1CC[C@@H](CO)O1)C(=O)O]
b. (R)-2-amino-3-(((2R,5R)-5-(hydroxymethyl)tetrahydrofuran-2-yl)methyl)thio)propanoic acid 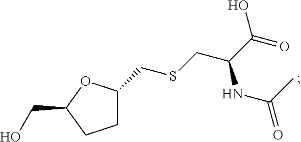 [SMILES: CC(=O)N[C@@H](CSC[C@@H]1CC[C@@H](CO)O1)C(=O)O]
and
c. (R)-2-amino-3-(((2S,5S)-5-hydroxymethyl)tetrahydrofuran-2-yl)methyl)thio)propanoic acid 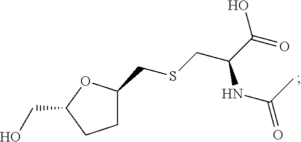 [SMILES: CC(=O)N[C@@H](CSC[C@H]1CC[C@H](CO)O1)C(=O)O]
Group IV.
a. ((2R,5S)-5-(fluoromethyl)tetrahydrofuran-2-yl)methanol 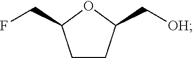
b. (2S,5S)-5-(fluoromethyl)tetrahydrofuran-2-yl)methanol and 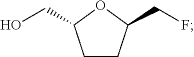
and
c. ((2R,5R)-5-(fluoromethyl)tetrahydrofuran-2-yl)methanol 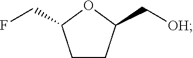
Group VII.
a. ((2R,5S)-5-(chloromethyl)tetrahydrofuran-2-yl)methyl 4-methylbenzenesulfonate 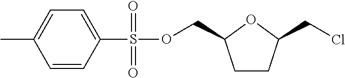 [SMILES: Cc1ccc(S(=O)(=O)OC[C@@H]2CC[C@H](CCl)O2)cc1]
b. ((2R,5R)-5-(chloromethyl)tetrahydrofuran-2-yl)methyl-4-methylbenzenesulfonate 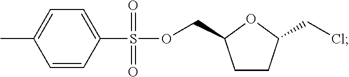 [SMILES: Cc1ccc(S(=O)(=O)OC[C@@H]2CC[C@@H](CCl)O2)cc1]
c. ((2S,5S)-5-chloromethyl)tetrahydrofuran-2-yl)methyl-4-methylbenzenesulfonate 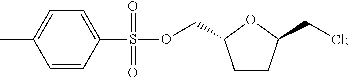 [SMILES: Cc1ccc(S(=O)(=O)OC[C@H]2CC[C@H](CCl)O2)cc1]
d. ((2R,5S)-5-chloromethyl)tetrahydrofuran-2-yl)methyl-4-methylbenzenesulfonate 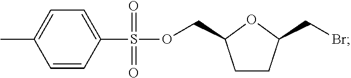 [SMILES: Cc1ccc(S(=O)(=O)OC[C@@H]2CC[C@H](CBr)O2)cc1]
e. (2R,5R)-5-(bromomethyl)tetrahydrofuran-2-yl)methyl-4-methylbenzenesulfonate 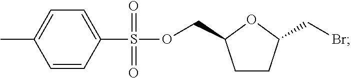 [SMILES: Cc1ccc(S(=O)(=O)OC[C@@H]2CC[C@@H](CBr)O2)cc1]
and
f. (2S,5S)-5-(bromomethyl)tetrahydrofuran-2-yl)methyl-4-methylbenzenesulfonate

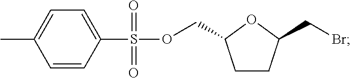 [SMILES: Cc1ccc(S(=O)(=O)OC[C@H]2CC[C@H](CBr)O2)cc1]

13. A secondary derivative compound made from said primary derivative compound of Group I in claim 12, the secondary derivative compound being selected from the group consisting of:
a. 4-(((2R,5S)-5-(hydroxymethyl)tetrahydrofuran-2-yl)methoxy)-4-oxobutan-1-aminium 2,2,2-trifluoroacetate 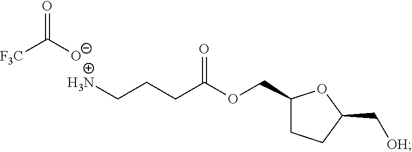 [SMILES: O=C([O-])C(F)(F)F ; [NH3+]CCCC(=O)OC[C@@H]1CC[C@H](CO)O1]
b. 4-(((2S,5S)-5-(hydroxymethyl)tetrahydrofuran-2-yl)methoxy)-4-oxobutan-1-aminium 2,2,2-trifluoroacetate 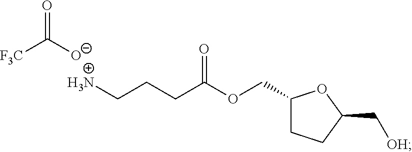 [SMILES: O=C([O-])C(F)(F)F ; [NH3+]CCCC(=O)OC[C@H]1CC[C@H](CO)O1]
and
c. 4-(((2R,5R)-5-(hydroxymethyl)tetrahydrofuran-2-yl)methoxy)-4-oxobutan-1-aminium 2,2,2-trifluoroacetate

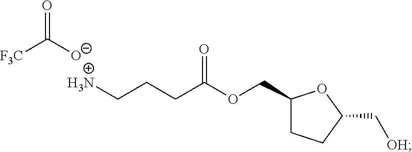 [SMILES: O=C([O-])C(F)(F)F ; [NH3+]CCCC(=O)OC[C@@H]1CC[C@@H](CO)O1]

14. (canceled)

15. (canceled)

16. (canceled)

17. A primary derivative compound made from THF-bismethylene disulfonate, the primary derivative compound being selected from at least one of the following groups:
Group I.
a. ((2R,5S)-tetrahydrofuran-2,5-diyl)-bis(methylene) bis(4-((tert-butoxycarbonyl)amino)butanoate) 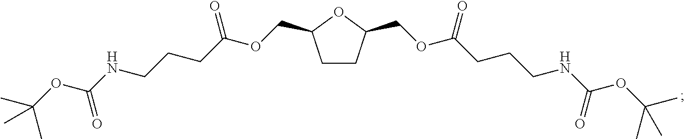 [SMILES: CC(C)(C)OC(=O)NCCCC(=O)OC[C@H]1CC[C@@H](COC(=O)CCCNC(=O)OC(C)(C)C)O1]
and
b. ((2S,5S)-tetrahydrofuran-2,5-diyl)-bis(methylene) bis(4-((tert-butoxycarbonyl)amino)butanoate) 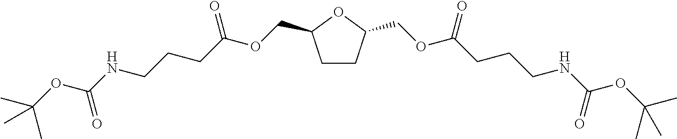 [SMILES: CC(C)(C)OC(=O)NCCCC(=O)OC[C@@H]1CC[C@@H](COC(=O)CCCNC(=O)OC(C)(C)C)O1]
Group II.
a. (2R,2′R)-3,3′-(((2R,5S)-tetrohydrofluoran-2,5-diyl)bis)methylene)-bis(sulfanediyl)-bis(2-aminopropanoic acid) 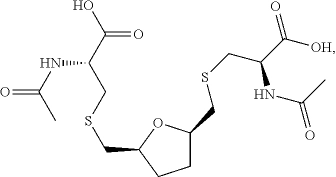 [SMILES: CC(=O)N[C@@H](CSC[C@H]1CC[C@@H](CSC[C@H](NC(C)=O)C(=O)O)O1)C(=O)O]
and
b. (2R,2′R)-3,3′-(((2R,5S)-tetrohydrofluoran-2,5-diyl)bis)methylene)-bis(sulfanediyl)-bis(2-aminopropanoic acid) 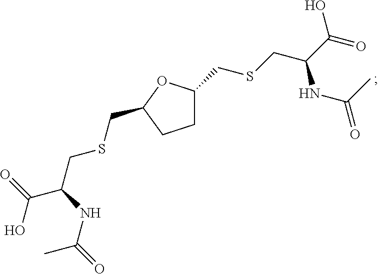 [SMILES: CC(=O)N[C@@H](CSC[C@@H]1CC[C@@H](CSC[C@@H](NC(C)=O)C(=O)O)O1)C(=O)O]
Group III.
c. (2R,5S)-2,5-bis(fluoromethyl)tetrahydrofuran 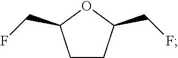
and
d. (2S,5S)-2,5-bis(fluoromethyl)tetrahydrofuran 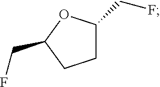
Group IV.
a phosphonium salt selected from the group consisting of:
e. ((2S,5R)-5-(((methylsulfonyl)oxy)methyl)tetrahydrofuran-2-yl)methyl)triphenylphosphorium methanesulfonate 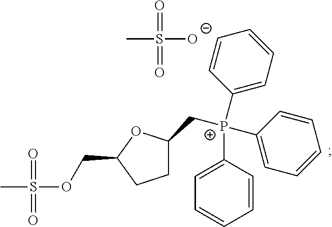
f. (((2R,5,R)-5-(((methylsulfonyl)oxy)methyl)tetrahydrofuran-2-yl)methyl)triphenylphosphorium methanesulfonate 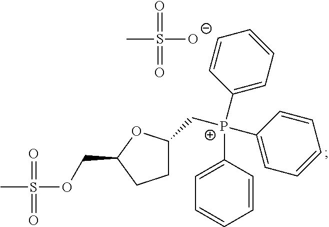
and
g. (2S,5S)-5-(((methylsulfonyl)oxy)methyl)tetrahydrofuran-2-yl)methyl)triphenylphosphorium methanesulfonate

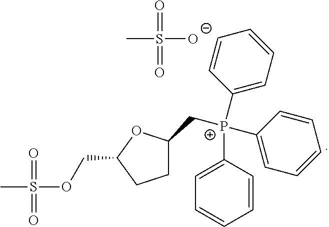

18. A secondary derivative compound made from said primary derivative compound of Group I in claim 17, the secondary derivative compound being at least one of:
a. 4,4′-((((2R,5S)-tetrahydrofuran-2,5-diyl)bis(methylene))bis(oxy))bis(4-oxobutan-1-aminium) 2,2,2-trifluoroacetate 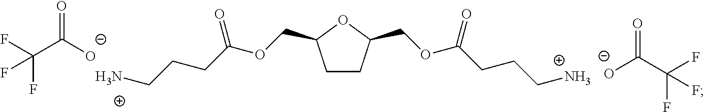 [SMILES: O=C([O-])C(F)(F)F ; [NH3+]CCCC(=O)OC[C@H]1CC[C@@H](COC(=O)CCC[NH3+])O1 ; O=C([O-])C(F)(F)F]
and
b. 4,4′-((((2S,5S)-tetrahydrofuran-2,5-diyl)bis(methylene))bis(oxy))bis(4-oxobutan-1-aminium) 2,2,2-trifluoroacetate

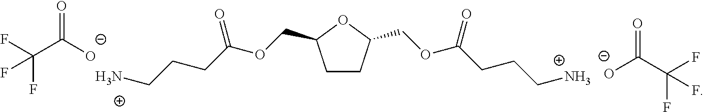 [SMILES: O=C([O-])C(F)(F)F ; [NH3+]CCCC(=O)OC[C@@H]1CC[C@@H](COC(=O)CCC[NH3+])O1 ; O=C([O-])C(F)(F)F]

19. (canceled)

20. (canceled)

21. (canceled)

22. (canceled)

23. (canceled)

24. (canceled)

25. A primary derivative compound made from furan-bismethylene monosulfonate, the primary derivative compound being at least one of:
a. (5-((benzylthio)methyl)furan-2-yl)methanol, 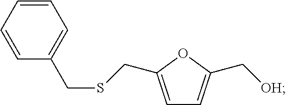 [SMILES: OCc1ccc(CSCc2ccccc2)o1]
and
b. (5-(fluoromethyl)furan-2-yl)methyl methanesulfonate,

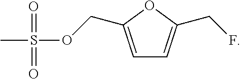

26. A primary derivative compound made from furan-bismethylene disulfonate, the primary derivative compound being at least one of:
a. N,N′-(furan-2,5-diylbis(methylene))-bis(1-phenyl methanamine), 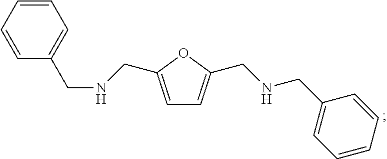 [SMILES: c1ccc(CNCc2ccc(CNCc3ccccc3)o2)cc1]
and
b. 2,5-bis(4-methoxybenzyl)furan, 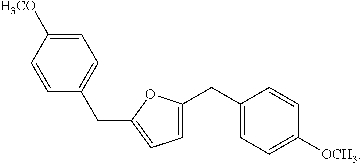 [SMILES: COc1ccc(Cc2ccc(Cc3ccc(OC)cc3)o2)cc1]
